(19) United States
(12) Patent Application Publication
(10) Pub. No.: US 2024/0065950 A1
(43) Pub. Date: Feb. 29, 2024

(54) AEROSOL DEVICE CONTAINING A COMPOSITION COMPRISING A SOLID FATTY ALCOHOL, A MONOALCOHOL AND A COMPRESSED GAS

(71) Applicant: L'Oreal, Paris (FR)

(72) Inventors: Guillaume RONCHARD, SAINT-OUEN (FR); Sophie BOUXIROT, SAINT-OUEN (FR)

(21) Appl. No.: 18/268,098

(22) PCT Filed: Dec. 16, 2021
(86) PCT No.: PCT/EP2021/086241

§ 371 Date: Jun. 16, 2023

(30) Foreign Application Priority Data
Dec. 18, 2020 (FR) FR2013747

(51) Int. Cl. A61K 8/04; A61K 8/34; A61Q 5/06; A61Q 5/12

(52) U.S. Cl. CPC A61K 8/046; A61K 8/342; A61Q 5/06; A61Q 5/12; A61K 2800/31

(57) ABSTRACT

The present invention relates to an aerosol device containing a composition, notably a cosmetic hair composition, comprising at least 5% by weight of one or more fatty alcohols, at least 20% by weight of one or more C₂-C₆ monoalcohols, a compressed gas and optionally one or more liquid fatty substances. The invention also relates to the use of the composition according to the invention for shaping and/or conditioning keratin fibres, in particular human keratin fibres such as the hair, and notably for defining the hair curls.

The present invention relates to an aerosol device containing a composition, notably a cosmetic composition, notably a hair composition, comprising at least 5% by weight of one or more fatty alcohols including at least one solid fatty alcohol, at least 20% by weight of one or more C₂-C₆ monoalcohols, at least one propellant chosen from compressed gases and optionally one or more liquid fatty substances other than liquid fatty alcohols.

The invention also relates to a process for shaping and/or conditioning keratin fibres, in particular human keratin fibres such as the head hair, comprising at least one step of applying to said fibres the composition according to the invention.

The invention lastly relates to the use of the composition sprayed from the aerosol device for shaping and/or conditioning keratin fibres, in particular human keratin fibres such as the hair, and in particular for providing softness, a smooth, non-tacky feel and sheen, for facilitating the disentangling of the hair, for defining the hair curls, and for providing control of the frizziness and volume of the hair. The aerosol format makes it possible to deliver a light mist that does not weigh the hair down and is easy to distribute on the hair.

Many individuals are unsatisfied with the appearance of their hair: in particular people who have curly hair may wish to maintain the curls but obtain better curl definition and better regularity, better movement and less volume and frizziness, even in humid conditions. Furthermore, some individuals find it difficult to control, define and/or style their curly hair.

Some individuals also wish for their hair to be soft and shiny.

There is a need to provide compositions that can simplify the styling routine, in particular in the case of people with curly hair.

In particular, there is a need to provide compositions which can maintain hair curls while at the same time giving them good definition and regularity, and which make it possible to obtain good control of the frizziness and volume of the hair, and which withstand humidity, while at the same time providing haircare, which may be persistent after shampooing several times.

In point of fact, the applicant has just discovered, surprisingly, that an aerosol device comprising a composition comprising the combination of one or more solubilized fatty alcohols, at least one C₂-C₄ monoalcohol and at least one compressed gas, makes it possible to solve the abovementioned problems.

One subject of the present invention is thus an aerosol device comprising a composition comprising:

at least 5% by weight, relative to the total weight of the composition, of one or more fatty alcohols, including at least one solid fatty alcohol, at least 20% by weight, relative to the total weight of the composition, of one or more C₂-C₆ monoalcohols, at least one propellant chosen from compressed gases, and optionally one or more liquid fatty substances other than liquid fatty alcohols, relative to the total weight of the composition.
Advantageously, the fatty alcohol(s) are solubilized in the composition.

The applicant has notably observed that the composition according to the invention makes it possible to obtain well-defined curls, good control of the frizziness and volume of the hair, and to give the hair good manageability. In addition, the composition according to the invention affords a hair conditioning effect and makes it possible in particular to give a pleasant cosmetic feel, notably a smooth, non-tacky feel, to facilitate the disentangling of the hair and to provide sheen. The composition provides neither a greasy effect nor a waxy effect, it does not transfer to the hands when the hands are passed through the hair and it provides good volume control. The use of an aerosol device enables a good distribution of the composition on the head of hair.

In addition, the compositions according to the invention are advantageously clear or transparent, hence an original visual effect for hair products of this type in transparent containers.

Furthermore, the composition according to the invention is distributed easily on the hair, notably on dry hair, in order to obtain conditioning and/or styling care.

Another subject of the invention is a method for shaping and/or conditioning keratin fibres, in particular human keratin fibres such as the hair, comprising at least one step of applying to said fibres a composition sprayed from an aerosol device according to the invention.

Other subjects, features, aspects and advantages of the invention will become even more clearly apparent on reading the description and the example which follows.

In the present description, the expression “at least one” is equivalent to the expression “one or more” and can be substituted for said expression; the expression “between” is equivalent to the expression “ranging from” and can be substituted for said expression, and implies that the limits are included.

The term “keratin fibres” means fibres of human or animal origin, such as head hair, body hair, the eyelashes, the eyebrows, wool, angora, cashmere or fur. According to the present invention, the keratin fibres are preferably human keratin fibres, more preferentially the hair.

The composition according to the invention is advantageously in the form of a clear to translucent, more preferentially clear, fluid. This property is notably advantageous in the case of transparent containers, in particular containers made of PET (polyethylene terephthalate).

The clarity of the composition according to the invention can be characterized by the measurement of its turbidity, by turbidimetry (in NTU units). In the context of the present invention, the turbidity measurements were carried out with a turbidimeter, model HI 88713-ISO from the company Hanna Instruments.

Preferably, the turbidity of the compositions according to the invention, measured at ambient temperature (25° C.) and atmospheric pressure, is less than 400 NTU units, more preferentially between 1 and 250 NTU units, even better still between 3 and 200 NTU units.

Fatty Alcohols

The composition according to the invention comprises at least 5% by weight of one or more fatty alcohols relative to the total weight of the composition, at least one of which is solid.

The term “fatty alcohol” means a non-glycerolated and non-oxyalkylenated, linear or branched, saturated or unsaturated aliphatic alcohol comprising from 6 to 40 carbon atoms and comprising at least one hydroxyl group and preferably containing from 8 to 30 carbon atoms, preferably from 12 to 24 carbon atoms, better still from 14 to 22 carbon atoms.

According to the invention, the fatty alcohols are preferably solubilized in the composition.

The term “solubilized fatty alcohol” means that the fatty alcohols do not precipitate from the composition and/or that they do not form crystals. When the composition is checked visually with the naked eye, it is homogeneous, single-phase, and clear or translucent.

The term “liquid” means a compound that is liquid at 25° C. and at atmospheric pressure (760 mmHg; i.e. 1.013×10⁵ Pa). Preferably, this compound has a viscosity of less than or equal to 2 Pa·s, preferably less than or equal to 1 Pa·s, more preferentially less than or equal to 0.1 Pa·s measured at a temperature of 25° C. and at a shear rate of 1 s⁻¹.

The term “solid” means a compound that is non-liquid at 30° C. and at atmospheric pressure (760 mmHg; i.e. 1.013×10⁵ Pa).

Solid Fatty Alcohols

The solid (non-liquid) fatty alcohols according to the invention are chosen from saturated or unsaturated, linear or branched alcohols comprising from 8 to 30 carbon atoms, preferably from 12 to 24 carbon atoms, better still from 14 to 22 carbon atoms.

More preferentially, the solid (non-liquid) fatty alcohols according to the invention are chosen from saturated linear alcohols comprising from 8 to 30 carbon atoms, preferably from 12 to 24 carbon atoms, better still from 14 to 22 carbon atoms.

Preferably, the solid fatty alcohols according to the invention are chosen from fatty alcohols having a melting point above 30° C., preferentially above 35° C.

Preferably, mention may for example be made of cetyl alcohol, stearyl alcohol, behenyl alcohol and myristyl alcohol, and mixtures thereof.

Liquid Fatty Alcohols

The liquid fatty alcohols of the invention may be saturated or unsaturated.

The saturated liquid fatty alcohols are preferably branched. They may optionally comprise in their structure at least one aromatic or non-aromatic ring. Preferably, they are acyclic.

More particularly, the saturated liquid fatty alcohols of the invention may be chosen from 2-octyldodecanol, isostearyl alcohol, 2-hexyldecanol, 2-butyloctanol and 2-undecylpentadecanol. Octyldodecanol is most particularly preferred.

The liquid unsaturated fatty alcohols contain in their structure at least one double or triple bond.

Preferably, they bear in their structure one or more double bonds. When several double bonds are present, they are preferably 2 or 3 in number and they can be conjugated or non-conjugated.

The liquid unsaturated fatty alcohols may be linear or branched. They may optionally comprise in their structure at least one aromatic or non-aromatic ring. Preferably, they are acyclic.

More particularly, the liquid unsaturated fatty alcohols of the invention are chosen from oleyl alcohol, linoleyl alcohol, linolenyl alcohol and undecylenyl alcohol, and mixtures thereof. Oleyl alcohol is particularly preferred.

In one preferred embodiment, the composition according to the invention comprises at least two distinct fatty alcohols, including at least one solid fatty alcohol.

Preferably, the composition according to the invention comprises at least one solid fatty alcohol and at least one liquid fatty alcohol.

According to this embodiment, the hair conditioning properties are improved, notably a smoother feel, and the hair is softer and more shiny.

Preferably, the ratio of the solid fatty alcohol/total fatty alcohol concentrations by weight is greater than 0.25 and preferably ranges from 0.3 to 1, preferentially from 0.3 to 0.9, more particularly from 0.35 to 0.8, or even from 0.4 to 0.7.

The composition according to the invention may comprise a total amount of one or more solid fatty alcohols ranging from 1% to 25% by weight, preferably from 2% to 20% by weight, and more preferentially from 3% to 15% by weight, relative to the total weight of the composition.

The composition according to the invention may comprise a total amount of one or more liquid fatty alcohols ranging from 1% to 20% by weight, preferably from 1.5% to 15% by weight, and more preferentially from 2% to 10% by weight, relative to the total weight of the composition.

The composition according to the invention may comprise a total amount of one or more fatty alcohols ranging from 5% to 25% by weight, preferably from 7% to 20% by weight, and more preferentially from 8% to 15% by weight, relative to the total weight of the composition.

C₂-C₆ Monoalcohols

The composition according to the present invention comprises at least 20% by weight of one or more C₂-C₆ monoalcohols.

The C₂-C₆, preferably C₂-C₄, monoalcohols are notably ethanol, isopropanol, tert-butanol, n-butanol. Preferably, the monoalcohol used is ethanol.

Preferably, the C₂-C₆ alcohol(s) are present notably in a total amount ranging from 20% to 95%, preferably from 50% to 90% by weight, in particular ranging from 60% to 85% by weight, or even from 65% to 85% by weight, relative to the total weight of the composition.

Propellants

The aerosol device according to the invention comprises a composition based on one or more propellants chosen from compressed gases.
Mention may be made, as examples of compressed gases, of air, nitrogen, carbon dioxide and mixtures thereof. Preferably, the compressed gas is carbon dioxide.

The propellant(s) is (are) generally present in a total amount ranging from 1% to 10% by weight, preferably from 2% to 5% by weight, relative to the total weight of the composition.

Liquid Fatty Substances

The composition according to the present invention may comprise one or more liquid fatty substances other than liquid fatty alcohols.

The term “fatty substance” means an organic compound that is insoluble in water at ordinary ambient temperature (25° C.) and at atmospheric pressure (760 mmHg, i.e. 1.013×10⁵ Pa), with a solubility in water of less than 5% by weight, preferably less than 1% by weight and even more preferentially less than 0.1% by weight. They have in their structure at least one hydrocarbon-based chain containing at least 6 carbon atoms. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane. The fatty substances of the invention are not etherified with oxyalkylenated or glycerolated groups. Preferably, they do not contain in their structure a carboxylic acid function COOH.

The term “hydrocarbon” means a compound comprising solely carbon and hydrogen atoms.

The term “fatty acid” means a compound comprising at least one linear or branched, saturated or unsaturated carboxylic acid function, comprising from 6 to 40 carbon atoms.

The term “fatty ester” means an ester of a fatty acid and/or of a fatty alcohol.

The term “fatty ether” means a compound comprising at least one linear or branched, saturated or unsaturated ether function, comprising at least one hydrocarbon-based group containing from 6 to 40 carbon atoms.

The term “non-silicone” means a compound of which the structure does not comprise any silicon atoms, and which therefore notably does not comprise any siloxane groups.

More particularly, the liquid fatty substances are non-silicone substances.

In particular, the fatty substances of the invention are not (poly)oxyalkylenated or (poly)glycerolated ethers.

The term “oil” means a “fatty substance” that is liquid at ambient temperature (25° C.) and at atmospheric pressure (760 mmHg or 1.013×10⁵ Pa).

More particularly, the fatty substances are chosen from C₆-C₁₆ liquid hydrocarbons, liquid hydrocarbons comprising more than 16 carbon atoms, non-silicone oils of animal origin, oils of triglyceride type of plant or synthetic origin, fluoro oils, liquid esters of a fatty acid and/or of a fatty alcohol other than triglycerides, liquid fatty ethers, dialkyl carbonates, and mixtures thereof.

It is recalled that the fatty esters and acids more particularly contain at least one saturated or unsaturated, linear or branched hydrocarbon-based group, comprising from 6 to 40 and better still from 8 to 30 carbon atoms, which is optionally substituted, in particular, with one or more hydroxyl groups (in particular 1 to 4). If they are unsaturated, these compounds may comprise one to three conjugated or non-conjugated carbon-carbon double bonds.

As regards the C₆-C₁₆ liquid hydrocarbons, they may be linear or branched, optionally cyclic, and are preferably alkanes. Examples that may be mentioned include hexane, cyclohexane, undecane, dodecane, tridecane or isoparaffins, such as isohexadecane, isodecane or isododecane, and mixtures thereof. C₆-C₁₆ isoparaffins are particularly preferred, more preferentially isododecane.

The linear or branched hydrocarbons of mineral or synthetic origin comprising more than 16 carbon atoms are preferably chosen from liquid paraffins, petroleum jelly, liquid petroleum jelly, polydecenes and hydrogenated polyisobutene such as Parleam® 4, and mixtures thereof.

The triglyceride oils of plant or synthetic origin are preferably chosen from liquid triglycerides of fatty acids comprising from 6 to 30 carbon atoms, such as heptanoic or octanoic acid triglycerides, or else, for example, sunflower oil, linseed oil, olive oil, safflower oil, argan oil, maize germ oil, wheat germ oil, soybean oil, marrow seed oil, grapeseed oil, sesame oil, hazelnut oil, apricot kernel oil, macadamia oil, arara oil, castor oil, avocado oil, caprylic/capric acid triglycerides, for instance those sold by the company Stéarineries Dubois or those sold under the names Miglyol® 810, 812 and 818 by the company Dynamit Nobel, shea butter, and mixtures thereof.

Fatty Esters

As regards the liquid esters of fatty acids and/or of fatty alcohols other than the triglycerides mentioned above and the fatty esters a) described above, mention may be made especially of esters of saturated or unsaturated, linear C₁-C₂₆ or branched C₃-C₂₆ aliphatic monoacids or polyacids and of saturated or unsaturated, linear C₁-C₂₆ or branched C₃-C₂₆ aliphatic monoalcohols or polyalcohols, the total carbon number of the esters being greater than or equal to 10.

Preferably, for the esters of monoalcohols, one at least of the alcohol or of the acid from which the esters of the invention are derived is branched and/or unsaturated.

Among the monoesters, mention may be made of dihydroabietyl behenate; octyldodecyl behenate; isocetyl behenate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl palmitate, isopropyl palmitate, 2-ethylhexyl palmitate, 2-octyldecyl palmitate, alkyl myristates such as isopropyl 2-octyldodecyl myristate, isobutyl stearate; 2-hexyldecyl laurate, and mixtures thereof.

Preferably, among the monoesters of monoacids and of monoalcohols, use will be made of isopropyl palmitates, alkyl myristates such as isopropyl myristate, isocetyl stearate, 2-ethylhexyl isononanoate, isodecyl neopentanoate, isononyl isononanoate, and mixtures thereof. Still within the context of this variant, use may also be made of esters of C₄-C₂₂ dicarboxylic or tricarboxylic acids and of C₁-C₂₂ alcohols, and esters of monocarboxylic, dicarboxylic or tricarboxylic acids and of C₂-C₂₆ dihydroxy, trihydroxy, tetrahydroxy or pentahydroxy alcohols.

Mention may notably be made of: diethyl sebacate; diisopropyl sebacate; diisopropyl adipate; di-n-propyl adipate; dioctyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; glyceryl trilactate; glyceryl trioctanoate; propylene glycol dioctanoate; neopentyl glycol diheptanoate; diethylene glycol diisononanoate; and mixtures thereof.

The composition may also comprise, as fatty ester, sugar esters and diesters of C₆-C₃₀ and preferably C₁₂-C₂₂ fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, especially alkyl derivatives, such as methyl derivatives, for instance methylglucose.

The esters of sugars and of fatty acids may especially be chosen from the group comprising the esters or mixtures of esters of sugars described previously and of linear or branched, saturated or unsaturated C₆-C₃₀, preferably C₁₂-C₂₂, fatty acids. If they are unsaturated, these compounds may comprise one to three conjugated or non-conjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, notably, oleopalmitate, oleostearate and palmitostearate mixed esters.

More particularly, use is made of monoesters and diesters and especially sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

According to one preferred embodiment, use may also be made of dialkyl carbonates, in particular di(C₁-C₁₂)alkyl carbonates, preferably di(C₄-C₁₂)alkyl carbonates such as notably dicaprylyl carbonate.

Fatty Ethers

The liquid fatty ethers may be chosen from dialkyl ethers, in particular di(C₁-C₁₂)alkyl ethers, preferably di(C₄-C₁₂)alkyl ethers, such as notably dicaprylyl carbonate.

Preferably, the composition according to the invention comprises one or more liquid fatty substances other than liquid fatty alcohols. According to one embodiment, the liquid fatty substance(s) is (are) chosen from C₆-C₁₆ alkanes, in particular branched C₈-C₁₆ alkanes, fatty ethers, dialkyl carbonates and mixtures thereof.

According to one preferred embodiment, the liquid fatty substance(s) are chosen from branched C₈-C₁₆ alkanes, di(C₁-C₁₂)alkyl ethers, di(C₁-C₁₂)alkyl carbonates and mixtures thereof, more preferentially from branched C₈-C₁₆ alkanes, di(C₁-C₁₂)alkyl ethers and mixtures thereof.

Even more preferentially, the liquid fatty substance(s) are chosen from di(C₁-C₁₂)alkyl ethers, preferably di(C₄-C₁₂)alkyl ethers, more particularly dicaprylyl ether.

The composition according to the invention may comprise the liquid fatty substance(s) other than liquid fatty alcohols in a total content ranging from 1% to 20% by weight, preferably from 2% to 15% by weight, preferentially from 3% to 12% by weight, relative to the total weight of the composition.

The composition according to the invention may be for cosmetic use. For the purposes of the invention, the term “cosmetic” means a composition of pleasant appearance, odour and feel. Preferably, the composition is for topical application to the skin and keratin fibres, and more especially to the scalp, head hair and eyelashes, and even better still to the head hair.

The composition according to the invention may be in the form of an alcoholic or aqueous-alcoholic solution of liquid or semi-liquid consistency.

When the composition according to the invention comprises water, the water content of the composition according to the invention is advantageously less than 10% by weight, preferably ranges from 1% to 8% by weight, in particular from 1% to 5% by weight, relative to the total weight of the composition.

Preferably, the composition according to the invention is anhydrous.

For the purposes of the present invention, the term “anhydrous” means that the water content is less than 1% by weight, preferably less than 0.5% by weight, and better still the composition is free of water. In particular, the composition according to the invention does not comprise any water added during its preparation, the residual water possibly being provided by the starting materials used for its preparation.

The composition according to the invention may further comprise one or more additional organic solvents, other than the C₂-C₆ monoalcohols and liquid fatty substances described above, for example chosen from polyols, especially those containing from 2 to 6 carbon atoms, for instance glycerol, propylene glycol, butylene glycol, pentylene glycol, hexylene glycol, dipropylene glycol or diethylene glycol; and polyol ethers such as 2-butoxyethanol, propylene glycol monomethyl ether, and diethylene glycol monoethyl ether and monomethyl ether; and mixtures thereof.

Preferably, the composition according to the invention comprises less than 10% by weight, preferentially less than 5%, more preferentially less than 2% by weight, better still less than 1% by weight of polyol(s), relative to the total weight of the composition. More particularly, the composition does not comprise any polyol.

The composition according to the invention may comprise one or more surfactants chosen from non-ionic, anionic, amphoteric or cationic surfactants, and mixtures thereof.

The composition according to the invention may also comprise one or more additional compounds chosen from natural or synthetic thickeners or viscosity regulators; UV-screening agents, fillers such as pearlescent agents, titanium dioxide, resins and clays, fragrances, peptizers, vitamins, acidic agents, alkaline agents, colorants, nacreous agents, opacifiers, film-forming polymers, fixing polymers, conditioning agents, solid fatty substances other than solid fatty alcohols, antioxidants and preserving agents.

Those skilled in the art will take care to select the optional additional compounds and the amount thereof such that they do not harm the properties of the composition of the present invention, notably the solubilization of the fatty alcohols.

These additives may be present in the composition according to the invention in an amount ranging from 0 to 20% by weight, relative to the total weight of the composition.

When the composition is aqueous, the pH of the composition according to the invention generally ranges from 2 to 12, preferably from 2.5 to 9, preferentially from 3 to 8 and better still from 4 to 7.

The pH of the composition may be adjusted to the desired value by means of basifying agents or acidifying agents that are customarily used. Among the basifying agents, examples that may be mentioned include aqueous ammonia, alkanolamines, and mineral or organic hydroxides. Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

The cosmetic composition may thus be rinsed off or left on after having been applied to the keratin materials, for example rinsed off with water, after an optional leave-on time. The composition is preferably a leave-on composition.

### Aerosol Device

The aerosol device according to the present invention comprising the composition is constituted of a container containing said composition and of a means for spraying said composition.

Preferably, the aerosol device according to the invention makes it possible to spray said composition in spray or mousse form, preferably in spray form.

The composition of use according to the invention is packaged under pressure, in an aerosol device, for example a monobloc device, which comprises a spraying means and a container.

The spraying means is generally constituted of a dispensing valve controlled by a dispensing head, itself comprising a nozzle through which the composition of the invention is sprayed. The valve may comprise an additional gas intake.

The container containing the pressurized composition may be opaque or transparent. It may be made of glass, polymer material or metal, and may optionally be coated with a protective varnish layer.

Another subject of the present invention is a method for shaping and/or conditioning keratin fibres, in particular human keratin fibres such as the hair, comprising at least one step of applying to said fibres a composition as described previously.

This or these steps of applying the composition according to the invention may optionally be preceded by at least one step of washing said fibres using a shampoo.

Moreover, this or these steps of applying the composition according to the invention may optionally be followed by a step of leaving the composition on, for example a leave-on time of 5 s to 15 minutes, notably 15 s to 5 minutes; then followed by an optional rinsing step, for example with water; and/or a drying step.

Preferably, the keratin fibres are not rinsed after the step(s) of applying the composition according to the invention. The expression “are not rinsed” means that no step of rinsing the keratin fibres is carried out for a period of less than 30 minutes, preferentially of 20 minutes, after the step of applying the composition according to the invention.

Preferably, the composition as described previously is used on curly hair, to define the curls.

The following examples serve to illustrate the invention without, however, being limiting in nature.

### EXAMPLES

### Example 1

Compositions 1 to 3 according to the invention were prepared from the following ingredients, the contents of which are indicated in the tables below (as g of active material).

TABLE 1

| | A1 | A2 | A3 |
|---|---|---|---|
| Ingredients | (invention) | (invention) | (invention) |
| Cetylstearyl alcohol | 8.54 | 5.34 | 5.34 |
| Oleyl alcohol | 2.13 | 5.34 | 5.34 |
| Dicaprylyl ether | 9.70 | 9.70 | 4.85 |
| CO₂ | 3 | 3 | 3 |
| Ethanol | qs 100 | qs 100 | qs 100 |

Before pressurization of the composition (without propellant), it was observed that the solid fatty alcohols were solubilized in the compositions according to the invention. No fatty alcohol crystals were observed. The compositions were transparent. The compositions were stable over time.
The fluid was produced by cold solubilization of the fatty alcohols and dicaprylyl ether in ethanol. The fluid was then pressurized in an aluminium or PET case with the compressed gas. A valve and a spray-type push button were added to ensure the dispensing of the product.

The application in the form of an aerosol spray enabled a homogeneous distribution.

It was observed that the hair treated with these compositions according to the invention was lightweight, smooth to the touch, soft, shiny and was in individual strands, it had good hairstyling properties (curl definition, control of frizziness and volume). The feel was neither greasy nor loaded.

### Example 2

The composition according to the invention was prepared from the following ingredients, the contents of which are indicated in the tables below (in g of active material).

TABLE 2

| | B1 | B2 |
|---|---|---|
| Ingredients | (invention) | (invention) |
| Cetylstearyl alcohol | 5.225 | 8.45 |
| Oleyl alcohol | 5.225 | 2.11 |
| Dicaprylyl ether | 9.5 | 4.8 |
| CO₂ | 5 | 4 |
| Water | — | 7.68 |
| Fragrance | q.s. | q.s. |
| Ethanol | qs 100 | qs 100 |

Before pressurization of the composition (without propellant), it was observed that the solid fatty alcohols were solubilized in the compositions according to the invention. No fatty alcohol crystals were observed. The compositions were transparent. The compositions were stable over time.

The fluid was produced by cold solubilization of the fatty alcohols and dicaprylyl ether in ethanol. The fluid was then pressurized in an aluminium or PET case with the compressed gas. A valve and a spray-type push button were added to ensure the dispensing of the product.

The application in the form of an aerosol spray enabled a homogeneous distribution.

The hair treated with these compositions according to the invention was lightweight, smooth to the touch, soft, shiny and was in individual strands, it had good hairstyling properties (curl definition, control of frizziness and volume). The feel was neither greasy nor loaded.

1-19. (canceled)

20. A composition comprising:
a) at least one fatty alcohol in an amount of at least 5% by weight, relative to the total weight of the composition, wherein the composition comprises at least one solid fatty alcohol,
b) at least one C₂-C₆ monoalcohol in an amount of at least 20% by weight, relative to the total weight of the composition,
c) at least one compressed gas propellant, and
d) optionally one liquid fatty substance other than liquid fatty alcohols.

21. The composition of claim 20, wherein the at least one solid fatty alcohol is chosen from cetyl alcohol, stearyl alcohol, behenyl alcohol, myristyl alcohol, or mixtures of two or more thereof.

22. The composition of claim 20, wherein the composition further comprises at least one liquid fatty alcohol.

23. The composition of claim 22, wherein the at least one liquid fatty alcohol is chosen from 2-octyldodecanol, isostearyl alcohol, 2-hexyldecanol, 2-butyloctanol, 2-undecylpentadecanol, oleyl alcohol, linoleyl alcohol, linolenyl alcohol, undecylenyl alcohol, or mixtures of two or more thereof.

24. The composition of claim 20, wherein the total amount of solid fatty alcohol(s) ranges from 1% to 25% by weight, relative to the total weight of the composition.

25. The composition of claim 22, wherein the total amount of liquid fatty alcohol(s) ranges from 1% to 20% by weight, relative to the total weight of the composition.

26. The composition of claim 20, wherein the ratio of the total amount of solid fatty alcohol(s) to the total amount of fatty alcohol(s) is greater than 0.25:1.

27. The composition of claim 20, wherein the total amount of fatty alcohol(s) ranges from 5% to 25% by weight, relative to the total weight of the composition.

28. The composition of claim 20, wherein the at least one fatty alcohol is soluble in the composition.

29. The composition of claim 20, wherein the at least one C₂-C₆ monoalcohol is chosen from ethanol, isopropanol, tert-butanol, n-butanol, or mixtures of two or more thereof.

30. The composition of claim 20, wherein the total amount of C₂-C₆ monoalcohol(s) ranges from 20% to 95% by weight, relative to the total weight of the composition.

31. The composition of claim 20, wherein the at least one propellant chosen from compressed gases is chosen from air, nitrogen, carbon dioxide, or mixtures of two or more thereof.

32. The composition of claim 20, wherein the total amount of propellant(s) ranges from 1% to 10% by weight, relative to the total weight of the composition.

33. The composition of claim 20, wherein the at least one liquid fatty substance other than liquid fatty alcohol is chosen from C₆-C₁₆ alkanes, fatty ethers, dialkyl carbonates, or mixtures of two or more thereof.

34. The composition of claim 20, wherein the total amount of liquid fatty substance(s) other than liquid fatty alcohols ranges from 1% to 20% by weight, relative to the total weight of the composition.

35. The composition of claim 20, wherein the composition further comprises e) at least one polyol in a total amount of less than 10% by weight, relative to the total weight of the composition.

36. The composition of claim 20, wherein the composition is anhydrous.

37. An aerosol device for spraying to the keratin fibers a composition comprising:
a) at least one fatty alcohol in an amount of at least 5% by weight, relative to the total weight of the composition, wherein the composition comprises at least one solid fatty alcohol,
b) at least one C₂-C₆ monoalcohol in an amount of at least 20% by weight, relative to the total weight of the composition,
c) at least one compressed gas propellant, and
d) optionally one liquid fatty substance other than liquid fatty alcohols.

38. A method for shaping and/or conditioning keratin fibers, comprising applying to said fibers a composition comprising:
a) at least one fatty alcohol in an amount of at least 5% by weight, relative to the total weight of the composition, wherein the composition comprises at least one solid fatty alcohol,
b) at least one C₂-C₆ monoalcohol in an amount of at least 20% by weight, relative to the total weight of the composition,
c) at least one compressed gas propellant chosen, and
d) optionally one liquid fatty substance other than liquid fatty alcohols.
